(19) United States
(12) Patent Application Publication
(10) Pub. No.: US 2023/0410990 A1
(43) Pub. Date: Dec. 21, 2023

(54) METHOD AND SYSTEM FOR AUTOMATED PATIENT WORK FLOW FOR MEDICAL DATA

(71) Applicant: Authentic, Inc., Bingham Farms, MI (US)

(72) Inventors: Kamil RAHME, Scottsdale, AZ (US); Jeffrey SANTOSO, Dural (AU)

(21) Appl. No.: 18/229,843

(22) Filed: Aug. 3, 2023

Related U.S. Application Data
(63) Continuation-in-part of application No. 17/942,054, filed on Sep. 9, 2022.
(60) Provisional application No. 63/278,960, filed on Nov. 12, 2021.

(51) Int. Cl. G16H 40/20; G16H 15/00; G16H 30/20

(52) U.S. Cl. CPC G16H 40/20; G16H 30/20; G16H 15/00

(57) ABSTRACT

In some implementations, the device may include receiving a check-in. In addition, the device may include determining, based on polling the medical service facility, that a patient service is complete. The device may include receiving, from the medical service facility, a medical image and an accession number associated with the patient service. Moreover, the device may include sending, to a medical analysis facility a request for a medical report. Also, the device may include receiving a message from the medical analysis facility indicating that the medical report is not ready. Further, the device may include receiving all medical reports generated by the medical analysis facility and an associated accession number with each medical report. In addition, the device may include identifying a medical report associated with the accession number. The device may include generating, based on the medical image and the medical report; a medical document. Moreover, the device may include generating a patient account with the medical document.

### CROSS-REFERENCES TO RELATED APPLICATION

This patent application is a Continuation-in-Part of U.S. Non-provisional application Ser. No. 17/942,054, filed Sep. 9, 2022, which claims the benefit of and priority to U.S. Provisional Patent Application Ser. No. 63/278,960, filed Nov. 12, 2021, the entire disclosures of which are incorporated herein by reference.

### FIELD OF THE DISCLOSURE

The present disclosure relates to an asynchronous medical patient data communication and management system. More particularly, the system of the present disclosure facilitates the secure communication of patient document files between healthcare providers with distinct internal IT networks.

### BACKGROUND OF THE DISCLOSURE

This section provides background information related to the present disclosure which is not necessarily prior art.

Hospital systems must manage large troves of sensitive user data. Such data has unique security regulations that lead to cumbersome security protocols. The data typically must be accessible by various individuals within a particular healthcare institution's internal network, such as doctors and staff. The data often must also be shareable with other parties, such as medical laboratories, clinics, and other healthcare systems. The increased security protocols, huge amount of data, and large number of people that must access the data leads to unique challenges faced by large healthcare institutions.

Often healthcare providers are required to share patient data in order to serve the patient. For example, scans of the patient (e.g., X-ray, MRI, CAT) are commonly analyzed by radiologists working off-site for a different healthcare entity. Without a secure means for transferring the data quickly, healthcare outcomes may suffer.

Hospitals are correctly cautious in granting full access to their internal database to external healthcare entities. Often access is limited to protect the privacy of patient information stored by the hospital. In order to grant access, hospitals often require paperwork to be filed, and associated review and granting of permission by hospital personnel.

Another concern that requires high data security is a need to comply with all current and future healthcare IT laws (such as HIPAA). HIPAA requires that hospitals limit access to their IT to authorized employees only. Sharing of health data between healthcare entities requires authentication between the systems. Violation of HIPAA policies can lead to considerable civil penalties.

Hospitals have attempted to utilize physical storage as a means of data exchange. Such means include a CD-ROM or portable memory device. In this instance, relevant data is loaded on the device and delivered to another healthcare entity. Such a transfer avoids granting remote access to patient data at all. However, this approach is very time consuming and dependent on mail services. The increasing size of data files is another concern, as physical media is generally not intended to transfer the scale of data required for high resolution 3D images.

In order to set up a secure connection between to healthcare providers, the process often depends on proprietary hardware being transferred from one provider to the other. If hardware is not required, then personnel from one entity must install and authenticate the necessary permission on the client device of the opposing healthcare entity. Both such approaches are typically time and resource intensive in order to create the network connection.

### SUMMARY OF THE DISCLOSURE

This section provides a general summary of the disclosure and is not intended to be interpreted as a comprehensive listing of its full scope or of all of its objects, aspects, features and/or advantages.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, a method may include receiving, in response to a patient arriving at a medical service facility, a check-in. A method may also include determining, based on polling the medical service facility, in response to the check-in, that a patient service is complete. The method may furthermore include receiving, in response to the patient service being complete, from the medical service facility, a medical image and an accession number associated with the patient service. The method may in addition include sending, to a medical analysis facility, based on the accession number, a request for a medical report. The method may moreover include receiving, from the medical analysis facility, a message from the medical analysis facility indicating that the medical report is not ready. The method may also include receiving, from the medical analysis facility, all medical reports generated by the medical analysis facility and an associated accession number with each medical report. The method may furthermore include identifying a medical report associated with the accession number. The method may in addition include generating, based on the medical image and the medical report; a medical document. The method may moreover include generating a patient account and associate the medical document with the patient account. Other embodiments of this aspect include corresponding computer systems, apparatus, and computer programs recorded on one or more computer storage devices, each configured to perform the actions of the methods.

Implementations may include one or more of the following features. A method where the medical image is retrieved from a PACS database. A method where the medical image is in a DICOM image format. A method where the medical service is one of a CT scan, X-ray, ultrasound, PET scan, MRI scan, mammogram, nuclear medicine image, and radiology scan. A method where the medical report is retrieved from a radiology information system or a hospital information system. A method may include: sending a notification to the patient indicating the medical document is available; generating a physician account and associate the medical document with the patient account; and sending the medical document to a physician associated with the patient. A method may include: verifying, in response to determining the medical report is associated with the accession number, the medical report is associated with the medical image. A method may include: deleting, in response to the medical report not being associated with the accession number, the medical report.

Implementations of the described techniques may include hardware, a method or process, or a computer tangible medium.

Other technical features may be readily apparent to one skilled in the art from the following figures, descriptions, and claims.

### DRAWINGS

The drawings described herein are for illustrative purposes only of selected embodiments and not all possible implementations thereof such that the drawings are not intended to limit the scope of the present disclosure.

FIG. 1 is a block diagram of an example computing environment, in accordance with the present disclosure.

FIG. 2 is a block diagram illustrating an example workflow of a medical document request fulfillment, in accordance with the present disclosure.

FIG. 3 is a flow chart of a process for managing medical document transfer at the request of a patient/physician, according to an example of the present disclosure.

FIG. 4 is a flow chart of a process, according to an example of the present disclosure.

FIG. 5 is a flow diagram illustrating an example workflow for managing a patient/physician request for medical images and medical reports, in accordance with the present disclosure.

FIG. 6 an example computing system, in accordance with the present disclosure.

Corresponding reference numerals indicate corresponding parts throughout the several view of the drawings.

### DETAILED DESCRIPTION OF THE EXAMPLE EMBODIMENT

Example embodiments of a patient document management system embodying the teachings of the present disclosure will now be described more fully with reference to the accompanying drawings. However, the example embodiments are only provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that the example embodiments may be embodied in many different forms that may be combined in various ways, and that neither should be construed to limit the scope of the disclosure. In some example embodiments, well-known processes, well-known device structures, and well-known technologies are not described in detail.

The systems and methods described herein are directed to automating document workflows associated with medical facilities. For example, a patient may require scans suggested by their physician. The patient may schedule an appointment with a medical service facility through their scheduling system which will store the appointment and track the check-in time of the patient. The patient may arrive at the medical service facility at their appointed time in order to receive the medical service (e.g., X-ray, MRI, CAT scan, or any appropriate medical service). The medical service facility's scheduling system may track the check-in time of the patient as well as when the medical service has been completed. The patient scan, generated by the patient service, may be stored by a medical image (e.g., PACS) system of the medical facility. The medical image may require further analysis by an analyzing physician that is associated with the medical service facility or a separate medical analysis facility (e.g., radiologist). The analyzing physician may draft a medical report based on his analysis of the medical image and upload it to a medical report system.

In some instances, the medical scheduling system, the medical image system, and the medical report system are not directly linked, which requires a human operator to manage communications between them. The systems and methods described herein automates the process of managing these documents across all the medical systems described above. A patient having access to the systems and methods described herein may receive their medical documents without interaction of a human user to manage the communication between the isolated medical systems.

In view of the foregoing, a method is desired that allows a healthcare entity to create a secure authenticated connection with another entity for the purpose of transferring patient files that only needs downloading and installing of software at the client device.

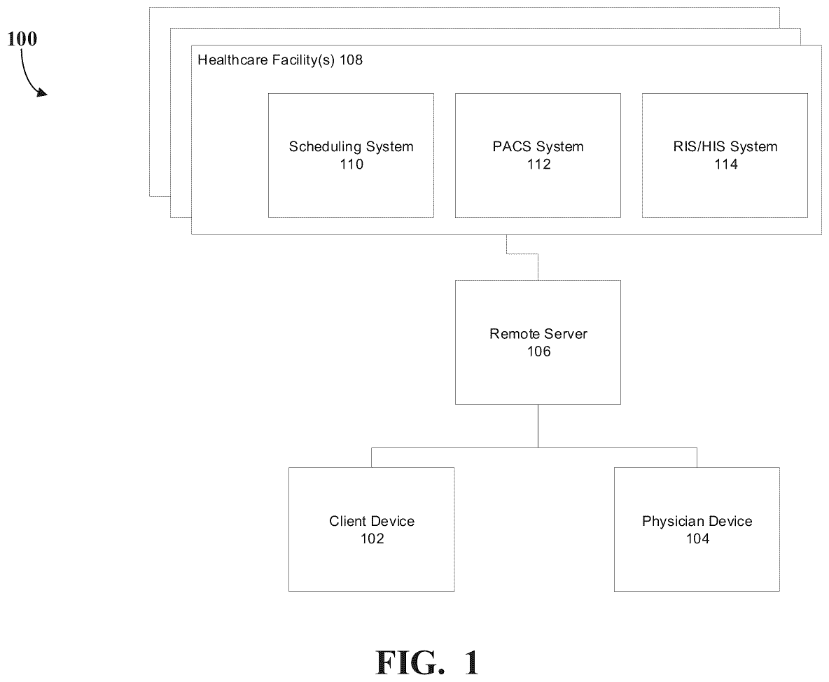

FIG. 1 is a block diagram of an example computing environment 100, in accordance with the systems and methods described herein. The computing environment 100 may include a client device 102 which may be used by the patient to request medical documents and access those documents through a patient account. The client device may execute the instructions found in a data object which is configured to install system component on the client device. The client device 102 may communicate at least one device property to a remote server 106. The remote computing server 106 may use the received device property in order to determine if the device is already registered to the system in order to authenticate the connection. The client device may be a computer having at least a processor and running an operating system (e.g., MS WINDOWS, APPLE OSX, CHROME OS, ANDROID, LINUX, APPLE IOS). The client device 102 may include a user interface which allows a user to input commands. The user interface may be based on mouse and keyboard inputs, touch based inputs, or any other applicable user interface input method.

The computing environment 100 may include a physician device 104, similar to that of client device 102, which may allow a physician access to medical documents associated with their patients. The client device 102 and physician device 104 may be connected to the remote server 106 via a network. The remote server 106 may distribute a data object which installs an application program interface (API) on any device in the computing environment 100. The API may allow access to databases of devices in the computing environment 100 and authenticate transfer of medical documents through the network. The remote server 106 may be one or many computing devices connected via a network. In some embodiments, the remote server 106 may operate as an API hosted a computing device associated with the healthcare facility.

The computing environment 100 may further include at least one healthcare facility device 108. The healthcare facility device 108 may be a single device or many devices connected via a network to facility the computing needs of a healthcare facility. The healthcare facility device 108 may include a healthcare scheduler 110. The healthcare scheduler 110 may handle scheduling functions of the healthcare facility including, but not limited to, scheduling appointments, confirming check-ins, scheduling medical services, confirming completion of medical services, or any appropriate scheduling operation for a medical facility.

The healthcare computing device 108 may further include a medical image system 112. The medical image system 112 may be a patient archiving communication system (PACS). The medical image system 112 may store medical images in a medical image format such as the digital image and communication in medicine (DICOM). The DICOM images are generated by a scanning modality such as an x-ray or MRI capture device. The patient is scanned by a medical image device and a medical image is created in a DICOM format. The medical image system 112 stores the image in the local data storage of the healthcare entity. The local data storage is a non-transitory computer readable medium. In some embodiments, requests for medical images made by a patient and or physician can be accessed from the medical image system 112 by the API.

The healthcare facility device 108 may further include a medical reporting system 114 such as a hospital information system (HIS) and/or a radiology information system (RIS). The medical reporting system 114 may manage medical reports generated by analyzing physicians such as, but not limited to, radiologists. In some embodiments, the API may access reports generated by analyzing physicians when they are uploaded to the medical report system.

In some embodiments, the patient may schedule a scan from a first healthcare facility which is analyzed by an analyzing physician in a second medical facility. The API may be configured to access and coordinate document sharing between multiple healthcare facilities.

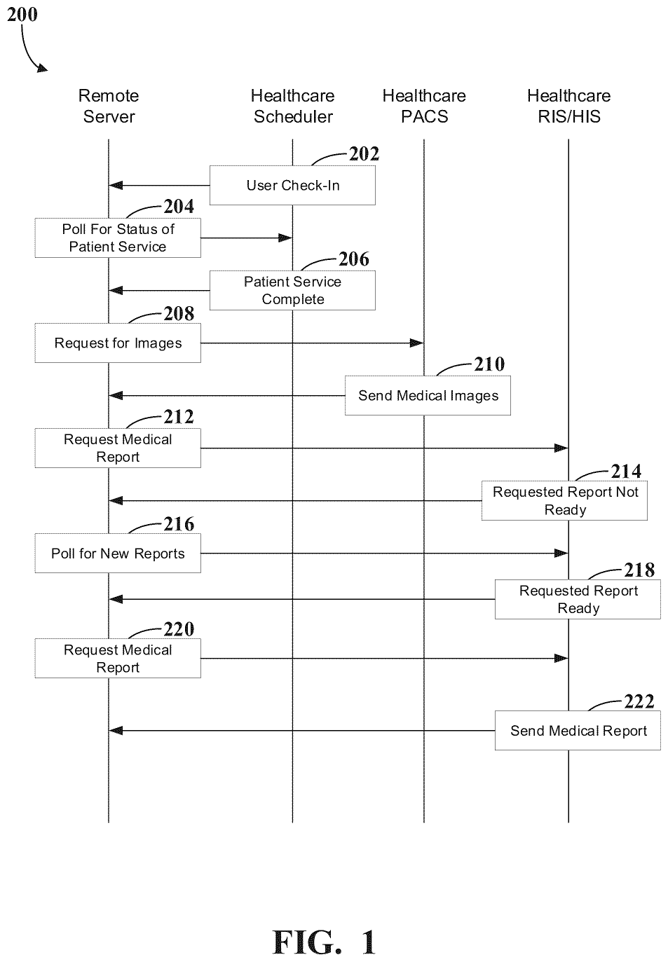

FIG. 2 is a block diagram illustrating an example workflow 200 of a medical document request fulfillment, in accordance with the systems and methods described herein. In some embodiments the workflow 200 may be executed on the computing environment 100, by a processor 604.

At 202, in response to a patient arriving at the healthcare facility, the healthcare scheduler 110 sends to the remote server 106 an indication of a user check-in. For example, when a patient arrives at the healthcare facility for their healthcare service appointment, they may check in with a front desk receptionist and/or a check-in kiosk. The check-in may be registered with the healthcare scheduler 110 which then sends an indication of the check-in to the remote server 106 via the preinstalled API.

At 204, the remote server 106 begins polling the healthcare scheduler 110 for updates regarding the status of the patient. For example, while the patient is waiting for their medical service, the healthcare scheduler 110 may return a “waiting” response. In a further example, while the patient is receiving the healthcare service, the healthcare scheduler 110 may return an “in progress” response. In some embodiments, the remote server 106 may provide the cloud support for the systems and methods described herein. All healthcare facilities which are subscribed to the systems and methods described herein will have their own computing device(s) that facilitate communication with the remote server 106. Different healthcare facilities 108 may communicate with one another, the communication may be facilitated by the remote sever 106.

At 206, the healthcare scheduler 110 may return a “service complete” response, in response to the medical service being complete and an indication of that completeness being registered by the healthcare scheduler 110. For example, when the user completes a scan and a medical image is generated based on the scan, the healthcare scheduler 110 may register that the medical service is complete. In some embodiments, the generation of the medical image is the indication based on which the healthcare scheduler 110 registers that the service is complete. In some embodiments, the indication that the medical service is complete is based on an input by a physician and/or technician associated with the medical service.

At 208, in response to the polling request returning a “service complete” response, the remote server 106 may send a medical image request to the medical image system 112. For example, when the patient medical service is complete and a medical image is generated, the remote server 106 may send a request to the healthcare image system for that medical image. In some embodiments, the API will send command inputs for the medical image system 112 to transfer DICOM images and execute commands such as C-Move, and C-Find.

At 210, in response to the medical image request by the remote server 106, the medical image system 112 may send, via a network, the medical image to the remote server 106. In some embodiments, the medical image is stored at the remote server 106. In some embodiments, the medical images are stored temporarily while the remote server 106 awaits further medical documents. The API may encrypts messages before they depart and decrypts them when they arrive to the remote sever 106.

In response to completeting the medical document request by the patient, the remote server 106 may discard/delete the locally stored medical documents. In some embodiments, the medical images may be stored by the healthcare facility until the medical image and the medical report associated with the medical image are both available and communicate all relevant documents to the remote server 106. In some embodiments, the remote server does not store any medical documents, instead transfers the medical documents from the medical facility to the client/physician device upon authorization by the remote server 106.

At 212, in response to receiving the medical images, the remote server 106 may request an associated medical report from the medical report system 114. For example, when a patient's scan is complete and the patient document request includes a request for a related medical document, the remote server 106 may begin polling the medical report system 114 for an associated medical document. In some embodiments, polling refers to the checking of the status of a computing device, via a network, in a repeated cycle. In some embodiments the polling request may be reiterated until a desired response is received. The polling request may be repeated a predetermined number of times and after a predetermined amount of time.

At 214, the medical report system 114 may indicate that the associated medical report is not yet ready. For example, when a medical image is generated and assigned for analysis by an analyzing physician, the physician may require time to analyze and draft the medical report. In some embodiments, the medical report system 114 may return a “report in progress” status, or a “report not yet started” status.

At 216, the remote server 106 polls the medical report system 114 for updates to the status of the medical report. In some embodiments, while the analyzing physician is completing the medical report, the medical report system 114 may make available new medical reports entered by an analyzing physician. The remote server 106 may compare the report with patient identification information to determine whether or not the newly entered medical report matches the medical scans of the patient.

At 218, the medical report system 114 may return, in response to the analyzing physician uploading the medical report, a “report ready” status. In response to receiving a report ready status, the remote server 106 may, at 220, send a medical report request for the medical report.

At 222, the medical report system 114 may, in response to the request, send the medical document to the remote server 106. In some embodiments, the medical report system 114 may allow access to the report by the remote server without sending the medical report. The access to the medical report may allow the patient to view the document stored at the medical report system 114 without transferring the file to the client device 102 or the remote server 106. In some embodiments, the transferring of the medical report and/or access to the medical report is facilitated by the API.

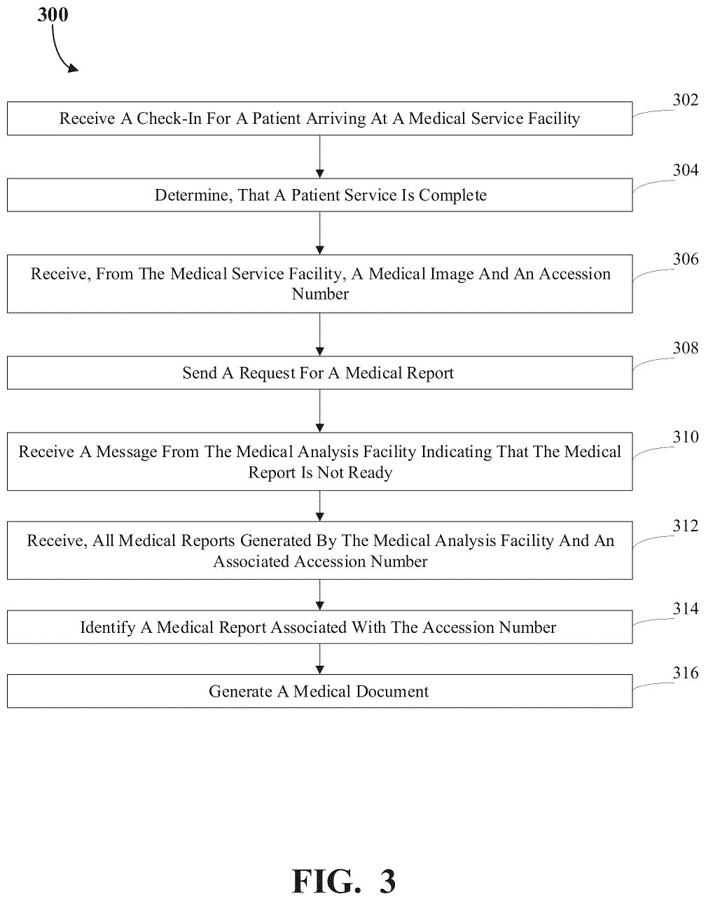

FIG. 3 is a flow chart of a process 300 for managing medical document transfer at the request of a patient/physician, according to an example of the present disclosure. According to an example, one or more process blocks of process 300 may be performed by a processor 604.

As shown in FIG. 3, process 300 may include receiving, in response to a patient arriving at a medical service facility, a check-in (block 302). For example, when a patient arrives at the healthcare facility for their healthcare service appointment, they may check in with a front desk receptionist and/or a check-in kiosk. The check-in may be registered with the healthcare scheduler 110 which then sends an indication of the check-in to the remote server 106 via the preinstalled API. The API may be installed on the computing devices of the medical facility with access to the healthcare scheduler 110, the medical image system 112, and the medical report system 114.

As in addition shown in FIG. 3, process 300 may include determining, based on polling the medical service facility, in response to the check-in, that a patient service is complete (block 304). For example, while the patient is waiting for their medical service, the healthcare scheduler 110 may return a “waiting” response or an “in progress” response based on the current status of the user. In a further example, while the patient is receiving the healthcare service, the healthcare scheduler 110 may return a “service complete” response. For example, when the user completes a scan and a medical image is generated based on the scan, the healthcare scheduler 110 may register that the medical service is complete. In some embodiments, the generation of the medical image is the indication based on which the healthcare scheduler 110 registers that the service is complete. In some embodiments, the indication that the medical service is complete is based on an input by a physician and/or technician associated with the medical service.

As also shown in FIG. 3, process 300 may include receiving, in response to the patient service being complete, from the medical service facility, a medical image and an accession number associated with the patient service (block 306). For example, the API may send, in response to the patient service being complete, the medical image produced by the medical service. In some embodiments, the medical facility may associate with the one or more medical images, an identifier known as an accession number. In some embodiments, the accession number provides a value which identifies the medical document within a document database and links them to the patient and/or medical service. In some embodiments, the value of the accession number may be numbers or alphanumeric characters.

As further shown in FIG. 3, process 300 may include sending, to a medical analysis facility, based on the accession number, a request for a medical report (block 308). For example, when a patient's scan is complete and the patient document request includes a request for a related medical document, the remote server 106 may begin polling the medical report system 114 for an associated medical document. In some embodiments, a medical facility may be part of the same medical facility where the medical service may be provided. In some embodiments the medical report may be identified based on identifying information of the patient. In some embodiments, the accession number may be generated by database storing the medical images.

As in addition shown in FIG. 3, process 300 may include receiving, from the medical analysis facility, a message from the medical analysis facility indicating that the medical report is not ready (block 310). For example, when a medical image is generated and assigned for analysis by an analyzing physician, the physician may require time to analyze and draft the medical report. In some embodiments, the medical report system 114 may return a “report in progress” status, or a “report not yet started” status. In some embodiments, the medical report is not associated with an accession number until it is complete.

As also shown in FIG. 3, process 300 may include receiving, from the medical analysis facility, all medical reports generated by the medical analysis facility and an associated accession number with each medical report (block 312). For example, while the analyzing physician is analyzing the one or more medical images associated with the medical service, other analyzing physicians may be uploading completed medical reports associated with other patients. In some embodiments, the process 300 may receive and/or be granted access to the newly uploaded reports. The reports may be analyzed by the API or the remote server 106 to determine whether or not the newly uploaded report is associated with the patient based on patient identification information and/or the accession number associated with the one or more medical images. In some embodiments, the remote server 106 may receive the newly uploaded medical report documents, and in response to determining they are not associated with the medical images and/or patient, delete the unwanted medical report document. In some embodiments, the newly uploaded medical report documents are not analyzed, instead metadata related to the medical report document are analyzed.

As further shown in FIG. 3, process 300 may include identifying a medical report associated with the accession number (block 314). For example, process 300 may identify one of the newly uploaded medical reports as having associated therewith the accession number which matches the accession number of the medical images. In some embodiments, once the medical report document is identified based on metadata related to the medical report document, the process 300 facilitates transfer/grants access of the medical document to the remote server 106.

As in addition shown in FIG. 3, process 300 may include generating, based on the medical image and the medical report; a medical document (block 316). For example, a medical service for a patient may be associated with one or more medical images and one or more medical reports. The medical images and medical reports may be merged into a medical document containing the one or more medical images and the one or more medical reports to generate a medical document associated with the medical service. In some embodiments, one or more medical images would maintain their DICOM file format and the one or more medical reports would maintain their original file format. In some embodiments, the format of the one or more medical images may be converted from a medical image format to an image format such as, but not limited to: tagged image file format (.TIFF), bitmap image file (.BMP), joint photographic experts group (.JPEG), graphics interchangeable format (.GIF), portable network graphics (.PNG), encapsulated post script (.EPS), raw image files (.RAW, .CR2, .NEF, .ORF, .SR2), or any appropriate image file format.

It should be noted that while FIG. 3 shows example blocks of process 300, in some implementations, process 300 may include additional blocks, fewer blocks, different blocks, or differently arranged blocks than those depicted in FIG. 3. Additionally, or alternatively, two or more of the blocks of process 300 may be performed in parallel.

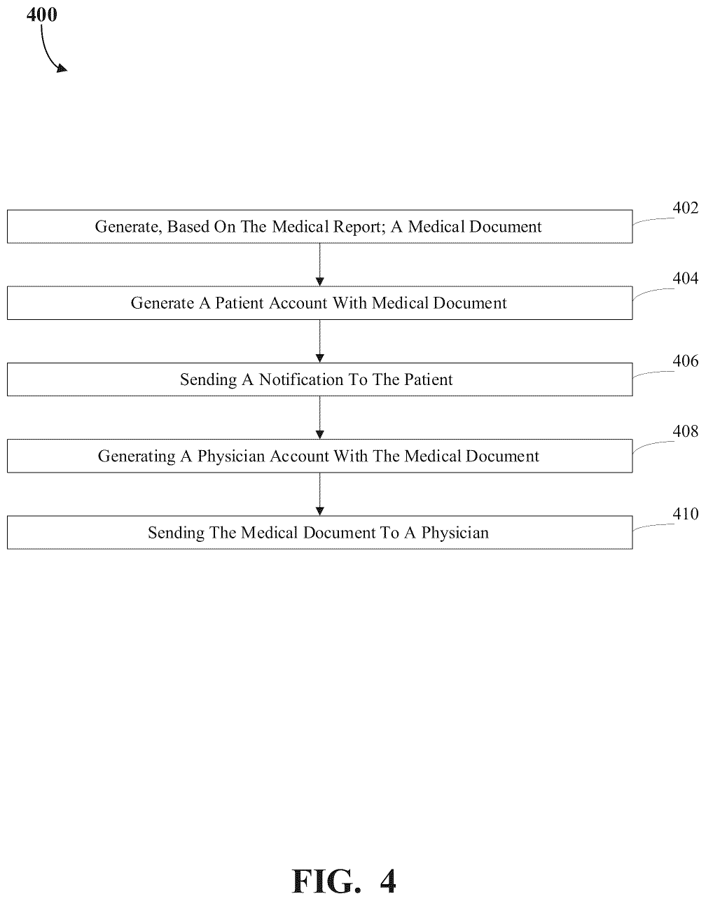

FIG. 4 is a flow chart of a process 400, according to an example of the present disclosure. According to an example, one or more process blocks of process 400 may be performed by the processor 604.

As shown in FIG. 4, process 400 may include generating, based on the medical image and the medical report; a medical document (block 402). For example, a medical service for a patient may be associated with one or more medical images and one or more medical reports. The medical images and medical reports may be merged into a medical document containing the one or more medical images and the one or more medical reports to generate a medical document associated with the medical service. In some embodiments, one or more medical images would maintain their DICOM file format and the one or more medical reports would maintain their original file format. In some embodiments, the format of the one or more medical images may be converted from a medical image format to an image format such as, but not limited to: tagged image file format (.TIFF), bitmap image file (.BMP), joint photographic experts group (.JPEG), graphics interchangeable format (.GIF), portable network graphics (.PNG), encapsulated post script (.EPS), raw image files (.RAW, .CR2, NEF, .ORF, .SR2), or any appropriate image file format.

As in addition shown in FIG. 4, process 400 may include generating a patient account and associate the medical document with the patient account (block 404). For example, process 300 may generate a patient account accessible by a patient and/or physician via a network by the client device 102 and/or the physician device 104. In some embodiments the patient account may be require registering the patient with patient identifying information, and creation of a password. The patient may login into the patient account using the password. The patient account may allow for downloading of the patient medical document and/or allow access for viewing of the patient medical document. In some embodiments, the medical document may be available for access to the user for a predetermined amount of time (e.g., hours, days).

As in addition shown in FIG. 4, process 400 may include sending a notification to the patient indicating the medical document is available (block 406). For example, process 400 may send a notification to the physician via the client device 102. In some embodiments, the notification may come in the form of, but not limited to, an email, social media message, voice over IPO, instant messaging, internet chat relay, video message, text message (SMS), video conferencing, application notification, RSS feed, blog post, message boards, or any appropriate form of device to device communication.

As in addition shown in FIG. 4, process 400 may include generating a physician account and associate the medical document with the patient account (block 408). For example, process 400 may generate an account for a physician where medical documents of patient associated with that physician will become accessible based on the systems and methods described above. In some embodiments, a physician account may be accessed by the physician as well as medical staff working with the physician. In some embodiments, the physician account may registered multiple log-in credentials for the physician as well as the staff of the physician. Physician staff include, but are not limited to: nurses, secretaries, administrators, physician assistants, technicians, and any other qualified medical personnel.

As in addition shown in FIG. 4, process 400 may include sending the medical document to a physician associated with the patient (block 410). For example, process 400 may be configured to allow access and/or transmit the medical document to the physician through the physician account. In some embodiments, the physician may access the physician account via a network by the physician device 104. In some embodiments the physician account may be require registering the patient with patient identifying information, and creation of a password. The patient may login into the patient account using the password. The physician account may allow for downloading of the patient medical document and/or allow access for viewing of the patient medical document. In some embodiments, the medical document may be available for access to the user for a predetermined amount of time (e.g., hours, days).

It should be noted that while FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

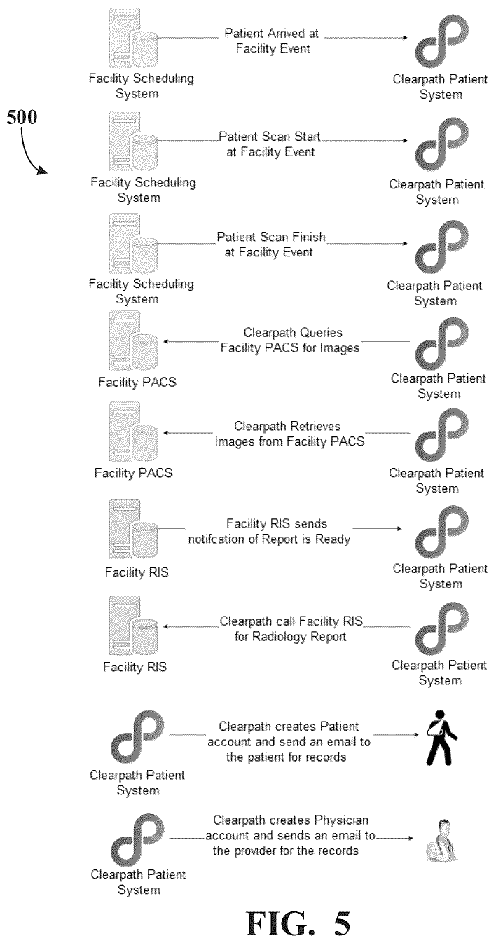

FIG. 5 is a flow diagram illustrating an example workflow 500 for managing a patient/physician request for medical images and medical reports, in accordance with the present disclosure. The steps of workflow 500 may be executed by the processor 604.

At 502, the healthcare scheduler 110 of a medical facility may provide a medical service to a patient sends the remote server 106 an indication that the patient has arrived at the facility. For example, when a patient arrives at the healthcare facility for their healthcare service appointment, they may check in with a front desk receptionist and/or a check-in kiosk. The check-in may be registered with the healthcare scheduler 110 which then sends an indication of the check-in to the remote server 106 via the preinstalled API. The API may be installed on the computing devices of the medical facility with access to the healthcare scheduler 110, the medical image system 112, and the medical report system 114.

At 504, the healthcare scheduler 110 may send an indication to the remote server 106 that the medical service has started. For example, while the patient is waiting for their medical service, the healthcare scheduler 110 may return a “waiting” response or an “in progress” response based on the current status of the user. In a further example, while the patient is receiving the healthcare service, the healthcare scheduler 110 may return a “service complete” response.

At 506, the healthcare scheduler 110 may send an indication to the remote server 106 that the medical service is complete. For example, when the user completes a scan and a medical image is generated based on the scan, the healthcare scheduler 110 may register that the medical service is complete. In some embodiments, the generation of the medical image is the indication based on which the healthcare scheduler 110 registers that the service is complete. In some embodiments, the indication that the medical service is complete is based on an input by a physician and/or technician associated with the medical service.

At 508, the remote server 106 may query the medical image system 112 for the one or more images generated by the medical service. For example, the API may send, in response to the patient service being complete, the medical image produced by the medical service. In some embodiments, the medical facility may associate with the one or more medical images, an identifier known as an accession number. In some embodiments, the accession number provides a value which identifies the medical document within a document database and links them to the patient and/or medical service. In some embodiments, the value of the accession number may be numbers or alphanumeric characters.

At 510, the medical image system 112 may send the one or more medical images to the remote server 106. In some embodiments, the medical image is stored at the remote server 106. In some embodiments, the medical images are stored temporarily while the remote server 106 awaits further medical documents. The API may encrypts messages before they depart and decrypts them when they arrive to the remote sever 106.

At 512, the medical report system 114 may send an indication to the remote server 106 that the medical report by the analyzing physician is available. In response to receiving a report ready status, the remote server 106 may, at 220, send a medical report request for the medical report.

At 514, the remote server 106 receive the medical report from the medical report system 114. In some embodiments, the medical report system 114 may allow access to the report by the remote server without sending the medical report. The access to the medical report may allow the patient to view the document stored at the medical report system 114 without transferring the file to the client device 102 or the remote server 106. In some embodiments, the transferring of the medical report and/or access to the medical report is facilitated by the API.

At 516, the remote server 106 may create a patient account which may allow the patient to access the one or more medical images and the medical report. The remote server 106 may further send a notification to the user which indicates the availability of the one or more medical images and the medical report at the patient account. For example, process 300 may generate a patient account accessible by a patient and/or physician via a network by the client device 102 and/or the physician device 104. In some embodiments the patient account may be require registering the patient with patient identifying information, and creation of a password. The patient may login into the patient account using the password. The patient account may allow for downloading of the patient medical document and/or allow access for viewing of the patient medical document. In some embodiments, the medical document may be available for access to the user for a predetermined amount of time (e.g., hours, days).

At 518, the remote server 106 may create a physician account which may allow the physician associated with the patient to access the one or more medical images and the medical report. The remote server 106 may further send a notification to the user which indicates the availability of the one or more medical images and the medical report at the physician account. For example, process 400 may generate an account for a physician where medical documents of patient associated with that physician will become accessible based on the systems and methods described above. In some embodiments, a physician account may be accessed by the physician as well as medical staff working with the physician. In some embodiments, the physician account may registered multiple log-in credentials for the physician as well as the staff of the physician. Physician staff include, but are not limited to: nurses, secretaries, administrators, physician assistants, technicians, and any other qualified medical personnel.

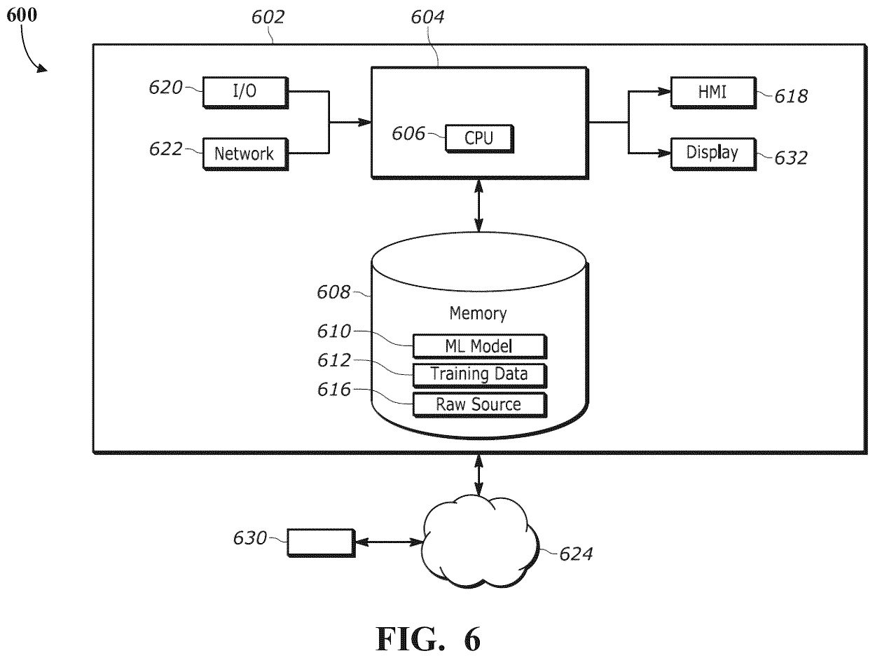

FIG. 6 an example computing system 602. Any of the client device 102, physician device 104, remote server 106, and healthcare facilities may comprise one or more computing systems 602. The computing system 602 may include at least one processor 604 that is operatively connected to a memory unit 608, which is a non-transitory computer readable medium. The processor 604 may be a multicore processor having multiple processors which may operate in parallel. The processor 604 may include one or more integrated circuits that implement the functionality of a central processing unit (CPU) 606. The CPU 606 may be a commercially available processing unit that implements an instruction stet such as one of the x86, ARM, Power, or MIPS instruction set families.

During operation, the CPU 606 may execute stored program instructions that are retrieved from the memory unit 608. The stored program instructions may include software that controls operation of the CPU 606 to perform the operation described herein. In some embodiments, the processor 604 may be a system on a chip (SoC) that integrates functionality of the CPU 606, the memory unit 608, a network interface, and input/output interfaces into a single integrated device. The computing system 602 may implement an operating system for managing various aspects of the operation.

The memory unit 608 may include volatile memory and non-volatile memory for storing instructions and data. The non-volatile memory may include solid-state memories, such as NAND flash memory, magnetic and optical storage media, or any other suitable data storage device that retains data when the computing system 602 is deactivated or loses electrical power. The volatile memory may include static and dynamic random-access memory (RAM) that stores program instructions and data. For example, the memory unit 608 may store a machine-learning model 610 or algorithm, a training dataset 612 for the machine-learning model 610, raw source dataset 616.

The computing system 602 may include a network interface device 622 that is configured to provide communication with external systems and devices. For example, the network interface device 622 may include a wired and/or wireless Ethernet interface as defined by Institute of Electrical and Electronics Engineers (IEEE) 802.11 family of standards. The network interface device 622 may include a cellular communication interface for communicating with a cellular network (e.g., 3G, 4G, 5G). The network interface device 622 may be further configured to provide a communication interface to an external network 624 or cloud.

The external network 624 may be referred to as the world-wide web or the Internet. The external network 624 may establish a standard communication protocol between computing devices. The external network 624 may allow information and data to be easily exchanged between computing devices and networks. One or more servers 630 may be in communication with the external network 624.

The computing system 602 may include an input/output (I/O) interface 620 that may be configured to provide digital and/or analog inputs and outputs. The I/O interface 620 may include additional serial interfaces for communicating with external devices (e.g., Universal Serial Bus (USB) interface).

The computing system 602 may include a human-machine interface (HMI) device 618 that may include any device that enables the system 600 to receive control input. Examples of input devices may include human interface inputs such as keyboards, mice, touchscreens, voice input devices, and other similar devices. The computing system 602 may include a display device 632. The computing system 602 may include hardware and software for outputting graphics and text information to the display device 632. The display device 632 may include an electronic display screen, projector, printer or other suitable device for displaying information to a user or operator. The computing system 602 may be further configured to allow interaction with remote HMI and remote display devices via the network interface device 622.

The system 600 may be implemented using one or multiple computing systems. While the example depicts a single computing system 602 that implements all of the described features, it is intended that various features and functions may be separated and implemented by multiple computing units in communication with one another. The particular system architecture selected may depend on a variety of factors. In some embodiments, the system 600 may be configured to perform the systems and methods described herein, using the system 600 and/or various classical computing algorithms.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in that particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or later, or intervening element or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to described various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for automating patient document transfer, the method comprising:
receiving, in response to a patient arriving at a medical service facility, a check-in;
determining, based on polling the medical service facility, in response to the check-in, that a patient service is complete;
receiving, in response to the patient service being complete, from the medical service facility, a medical image and an accession number associated with the patient service;
sending, to a medical analysis facility, based on the accession number, a request for a medical report;
receiving, from the medical analysis facility, all medical reports generated by the medical analysis facility and an associated accession number with each medical report;
identifying a medical report associated with the accession number;
generating, based on the medical image and the medical report, a medical document; and
generating a patient account and associate the medical document with the patient account.

2. The method of claim 1, wherein the medical image is retrieved from a PACS database.

3. The method of claim 1, wherein the medical image is in a DICOM image format.

4. The method of claim 1, wherein the medical service is one of a CT scan, X-ray, ultrasound, PET scan, MRI scan, mammogram, nuclear medicine image, and radiology scan.

5. The method of claim 1, wherein the medical report is retrieved from a radiology information system or a hospital information system.

6. The method of claim 1, further comprising:
sending a notification to the patient indicating the medical document is available;
generating a physician account and associate the medical document with the patient account; and
sending the medical document to a physician associated with the patient.

7. The method of claim 1, further comprising:
verifying, in response to determining the medical report is associated with the accession number, the medical report is associated with the medical image.

8. The method of claim 1, further comprising:
receive, from the medical analysis facility, a message from the medical analysis facility indicating that the medical report is not ready; and
deleting, in response to the medical report not being associated with the accession number, the medical report.

9. A device for automating patient document transfer comprising:
one or more processors configured to:
receive, in response to a patient arriving at a medical service facility, a check-in;
determine, based on polling the medical service facility, in response to the check-in, that a patient service is complete;
receive, in response to the patient service being complete, from the medical service facility, a medical image and an accession number associated with the patient service;
send, to a medical analysis facility, based on the accession number, a request for a medical report;
receive, from the medical analysis facility, a message from the medical analysis facility indicating that the medical report is not ready;
receive, from the medical analysis facility, all medical reports generated by the medical analysis facility and an associated accession number with each medical report;
identify a medical report associated with the accession number;
generate, based on the medical image and the medical report, a medical document; and
generate a patient account and associate the medical document with the patient account.

10. The device of claim 9, wherein the medical image is retrieved from a PACS database.

11. The device of claim 9, wherein the medical image is in a DICOM image format.

12. The device of claim 9, wherein the medical service is one of a CT scan, X-ray, ultrasound, PET scan, MRI scan, mammogram, nuclear medicine image, and radiology scan.

13. The device of claim 9, wherein the medical report is retrieved from a radiology information system or a hospital information system.

14. The device of claim 9, wherein the one or more processors are further configured to:
send a notification to the patient indicating the medical document is available;
generate a physician account and associate the medical document with the patient account; and
send the medical document to a physician associated with the patient.

15. The device of claim 9, wherein the one or more processors are further configured to:
verify, in response to determining the medical report is associated with the accession number, the medical report is associated with the medical image.

16. The device of claim 9, wherein the one or more processors are further configured to:
delete, in response to the medical report not being associated with the accession number, the medical report.

17. A device for automating patient document transfer comprising:
one or more processors configured to:
receive, in response to a patient arriving at a medical service facility, a check-in;
determine, based on polling the medical service facility, in response to the check-in, that a patient service is complete;
receive, in response to the patient service being complete, from the medical service facility, a medical image and an accession number associated with the patient service;
send, to a medical analysis facility, based on the accession number, a request for a medical report;
receive, from the medical analysis facility, all medical reports generated by the medical analysis facility and an associated accession number with each medical report;
identify a medical report associated with the accession number;
generate, based on the medical image and the medical report, a medical document; and
generate a patient account and associate the medical document with the patient account.

18. The device of claim 17, wherein the medical image is retrieved from a PACS database.

19. The device of claim 17, wherein the one or more processors are further configured to:
send a notification to the patient indicating the medical document is available;
generate a physician account and associate the medical document with the patient account; and
send the medical document to a physician associated with the patient.

20. The device of claim 17, wherein the one or more processors are further configured to:
verify, in response to determining the medical report is associated with the accession number, the medical report is associated with the medical image.
